(19) United States
(12) Patent Application Publication
(10) Pub. No.: US 2017/0196618 A1
(43) Pub. Date: Jul. 13, 2017

(54) METHOD FOR PRODUCING A FLAT ELECTRODE

(71) Applicant: OLYMPUS WINTER & IBE GMBH, Hamburg (DE)

(72) Inventors: Uwe SCHÖLER, Hoisdorf (DE); Mathias KRAAS, Haseldorf (DE); Hannes MIERSCH, Hamburg (DE)

(73) Assignee: OLYMPUS WINTER & IBE GMBH, Hamburg (DE)

(21) Appl. No.: 15/314,967

(22) PCT Filed: Jul. 3, 2015
(86) PCT No.: PCT/EP2015/001349

§ 371 Date: Nov. 30, 2016

(30) Foreign Application Priority Data
Jul. 17, 2014 (DE) 10 2014 010 791.4

(51) Int. Cl. A61B 18/14; B22F 7/02

(52) U.S. Cl. CPC A61B 18/14; A61B 18/1442; B22F 7/02; A61B 2018/00148

(57) ABSTRACT

A method produces a flat electrode of a high-frequency surgical instrument, which has in at least one layer height of metal and in at least one other layer height of ceramic, wherein the flat electrode is produced in the form of a green body and then sintered.

The invention relates to a method for producing a flat electrode according to the preamble of Claim 1.

High-frequency surgical instruments according to the preamble are known, for example, from U.S. Pat. No. 6,447,511 B1. FIGS. 12 and 13 of this published document show bipolar scissors for endoscopic purposes with two flat electrodes designed in the form of cutting blades that consist of metal, preferably ceramic, in different layer heights. The flat electrodes represented each have a central layer made of insulating material as well as an upper and a lower adjoining layer made of metal. Each of the two metal layers can be connected to another pole of a high-frequency source, so that these layers have different polarities in the two cutting blades and enable a current to flow between themselves through water or tissue, generating a cutting action. The other metal layer of the scissor blade in each case lies towards the contact surface between the cutting blades and is used primarily because metal can be ground to a sharper edge in comparison to insulating materials.

Another prior art according to the preamble is shown in DE 10 2007 054 438 A1. The flat electrode here is formed as a surgical vaporization electrode with an electrode head that has a working surface and is provided on the back side with a ceramic covering, in order to prevent the formation of hot plasma against the surrounding fluid.

The two cases involve a flat electrode that is coated with a layer of material, which consists of different material in different layer heights. “Layer heights” here denotes the distance from one of the surfaces of the flat electrode. In the known constructions, the flat electrode is constructed from layers that can be produced in different ways according to the prior art. Thus, for example, in the case of scissors, the cutting blade can be formed from different partial shells that are placed one on top of the other, wherein, for the connection, for example, a bonding occurs. Layers made of different materials can also be applied successively, for example, by galvanic deposition.

In the production of such flat electrodes, a number of problems arise. Bonding made from partial shells is not very durable, above all at higher temperatures, which are, however, unavoidable in the case of high-frequency surgical instruments. The resistance to rupturing also represents a major problem. Partial shells made of ceramic break very easily, for example. Moreover, it is difficult to sharpen scissor cutting blades, because the layered structure is commonly damaged during grinding.

Therefore, a great need exists for developments in the field of the methods according to the preamble.

This problem is solved by the features of the characterizing part of Claim 1.

According to the invention, the flat electrode is produced in the form of a green body and then sintered. “Green body” is understood to mean an unfired blank, which is still plastically deformable and is formed from the so-called feedstock, a mixture of ceramic powder or metal powder and a binder usually consisting primarily of a polymer. With a suitable binder, the feedstock can be sufficiently plastically deformable so as to be brought, for example, by injection molding, to the desired shape of the green body which is then possibly still plastic. After binder removal (removal of the binder) and sintering, the desired workpiece made of ceramic and/or metal is formed from the green body.

In the present field, sintering is not unknown, but it has been used only in partial steps of the production, for example, in the production of bonded partial shells. However, the present invention provides for producing the entire flat electrode first in the form of a green body and then sintering it. This results in the formation of an intimate connection between all the parts of the layered structure during the production of the green body. Next, the green body is sintered in this intimate connection. The result is a workpiece in which, in particular, the different materials are inseparably connected at the different layer heights. This also results, in particular, in improved mechanical strength, particularly resistance to rupturing and strength under thermal stresses. The durability increases enormously. The producibility is also considerably improved, since the production problems are shifted almost entirely to the work done on the green body. But it is much easier to work on the green body than on the finished sintered workpiece. The green body can be shaped easily, and, in the case of incorrect processing, it can even still be repaired.

A green body with layers that have different proportions of metal and ceramic is produced advantageously according to Claim 2 from green films which are applied one on top of the other and which are each produced from feedstocks with different proportions of metal and ceramic. As a result, the production is greatly simplified, since the green films can be prefabricated, for example, in larger batches.

Using green films, very thick-layered flat electrodes can be constructed, which, for example, consist only of metal, for the purpose of which green films with identical metal compositions are arranged one on top of the other. However, according to Claim 3, alternating films made of different materials are advantageously applied one on top of the other, making it possible to produce, for example, flat electrodes as are known from the printed documents mentioned at the start, which consist of metal or ceramic in different layer heights.

A very interesting possibility offered by sintering is the method according to Claim 4, wherein the base body has a grain mixture made of metal and ceramic, in which the mixing ratio changes with the layer height. Mixtures of metal and ceramic can be sintered satisfactorily. A flat electrode is formed in which the metal and ceramic proportions blend into one another with continuous transition. This results in particularly positive durability properties. The mixing ratio can here change continuously or preferably stepwise in a structure made of several films, in which the mixing ratio changes stepwise.

After the flat electrode has been produced, that is, after the sintering, it can be processed, for example, by drilling or grinding. However, this is difficult and expensive due to the hardness of the ceramic material. Therefore, according to Claim 5, the green body is advantageously brought to the desired shape before the sintering, so that processing steps after the sintering can be dispensed with. Green bodies can also consist of an easily shaped, kneadable material and can therefore very easily be shaped, cut, punched or processed in another manner. For this purpose, a temperature increase can be helpful, for example, when a thermoplastic binder is used.

In the drawings, the invention is represented in an exemplary and diagrammatic manner. In the drawings:

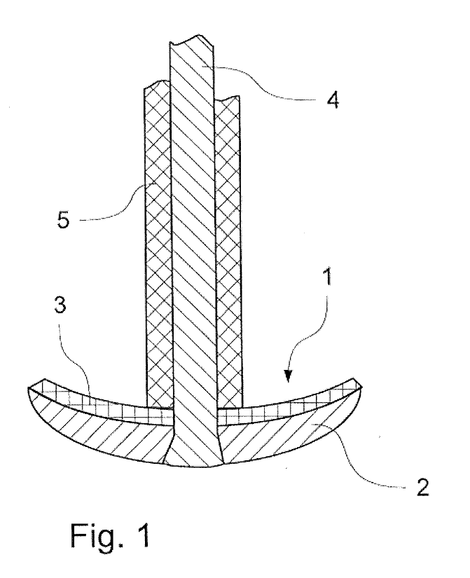

FIG. 1 shows a flat electrode of a vaporization instrument according to the invention in cross section,

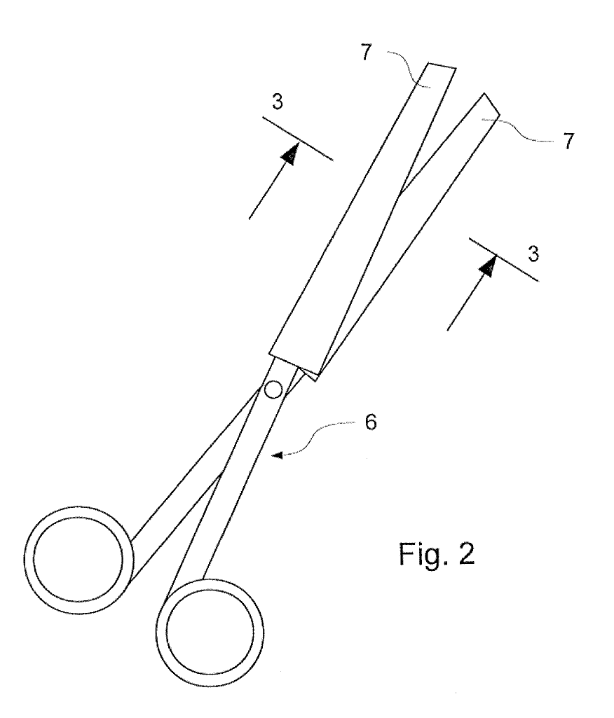

FIG. 2 shows a side view of bipolar scissors,

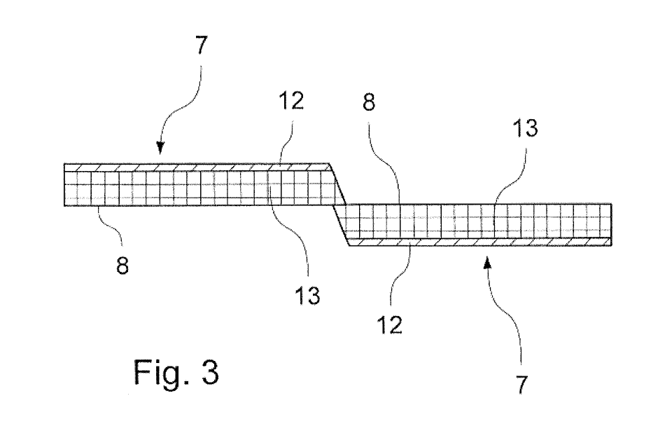

FIG. 3 shows a cross section along line 3-3 in FIG. 2,

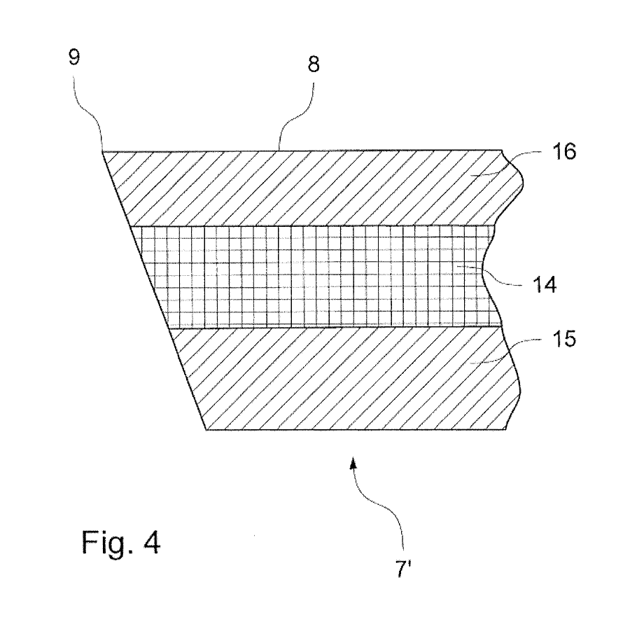

FIG. 4 shows a detail from FIG. 3 in a modified embodiment, and

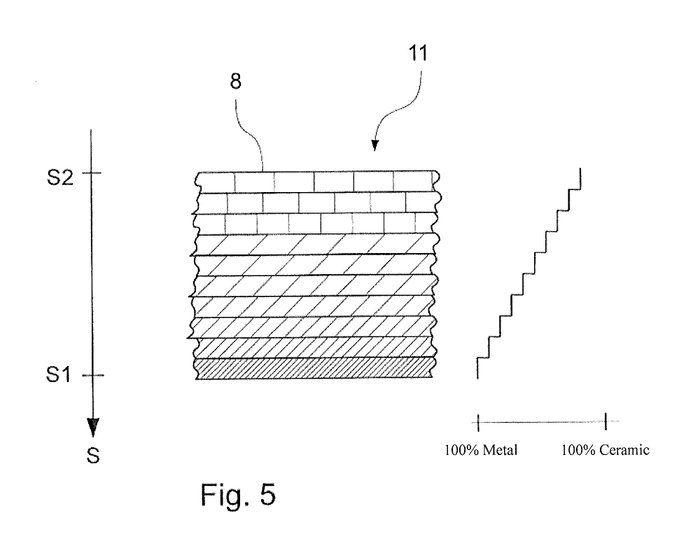

FIG. 5 shows a section of a flat electrode of another embodiment.

FIG. 1 shows a surgical vaporization electrode similar to the one explained in DE 10 2007 054 438 A1. The electrode arrangement represented in FIG. 1 comprises a flat electrode in the form of an electrode head 1, which, slightly curved, consists of a metal layer 2 and a ceramic back layer 3. A metal connection wire 4 passes through the flat electrode and is connected in an electrically conductive manner to the metal layer 2. The connection wire 4 is covered with a protective insulating sleeve 5 made of plastic, for example.

Known methods for the production of this electrode head, for example by bonding of prefabricated metal layers 2 with ceramic layers 3, are difficult, especially due to the small dimensions of only a few millimeters external diameter of the electrode head.

In addition, such an electrode is used in bipolar high-frequency application in a conductive fluid, which leads to extreme temperature stress. The layered structure of the flat electrode can then be destroyed very rapidly.

An additional problem area according to U.S. Pat. No. 6,447,511 B1, mentioned at the beginning, is represented in the embodiment of FIG. 2 with bipolar scissors 6, in which the flat electrodes are arranged in the form of cutting blades 7 that can be seen in detail in FIG. 3. The cutting blades 7 each consist of a ceramic layer 13 facing the cutting surface 8 and of a metal layer 12 adjoining said ceramic layer. Here too, problems of producibility arise.

These problems substantially relate to the question of how the metal layer 2 or 12 is to be connected to the ceramic layer 3 or 13.

The invention solves this problem by shifting the connection of the two layers to processing steps on a green body. The flat electrode 1 of FIG. 1, like the cutting blade 7 of FIG. 3, is produced in the form of a green body.

“Green body” is understood to mean the blank that is still plastically deformable and consists of a mixture of ceramic powder and/or metal powder and a binder. By firing or sintering, the desired workpiece is made from the green body.

The invention produces the flat electrode, that is to say the electrode head 1 of FIG. 1, or the cutting blade 7 of FIG. 3, in the form of a green body. Here, in each case, the metal layer 2 or 12 and the ceramic layer 3 or 13 can be produced separately in the form of a green film and combined by stacking one on top of the other to form the green body of the flat electrode. Green films made of the same material can be superposed in order to produce, for example, a very thick flat electrode made only of metal. Preferably, different materials, metal and ceramic as in the example shown, are used.

As shown in FIG. 1, a complicated, shell-shaped, rounded shaping of the flat electrode 1 can also be produced very simply by shaping the still-bendable green body, which, for example, can be brought to the desired shape very simply in a compression mold or by injection molding. The hole through which the connection wire 4 is passed can very simply be punched in the process. As a result, very expensive process steps that would have been required if the corresponding processing had occurred after the sintering are dispensed with.

FIG. 4 shows an enlarged detail in the area of the cutting edge of a scissor blade 7′, similar to the cutting blade 7 of FIG. 3. However, the layered structure is different. Here, not two layers, as in the case of FIG. 3, but three layers are used that can be arranged similarly to the case of above-cited U.S. Pat. No. 6,447,511 B1, namely with an insulation layer 14 in the center and with metal layers 15 and 16 adjoining at the top and at the bottom. In comparison to the construction of FIG. 3 with only two layers, the additional metal layer 16 on the cutting surface 8 gives an improved possibility of grinding the cutting edge 9 sharp, which would be more difficult in the case of the construction of FIG. 3, in which this edge is made of ceramic. There are also other reasons in favor of such a multi-layered structure, such as, for example, reasons pertaining to potential control.

FIG. 5 shows a section of an electrode 11 that is formed as a flat electrode. This can be, for example, a detail from the cutting blade 7 of FIG. 3. In comparison to the previous embodiments, one can see the essential difference, namely the material transition that changes stepwise with the layer height. Here, layer height S denotes the distance from the cutting surface 8.

In different layer heights, there are different concentrations of metal and ceramic in the material of the represented electrode 11. Layer height S1 is made entirely of metal, and S2 entirely of ceramic. In between, the mixture of ceramic particles and metal particles is modified stepwise.

The stairs represented in FIG. 5 next to the electrode 11 illustrate that the mixture of ceramic particles and metal particles changes stepwise in the electrode 11 from S1 to S2 from 100% metal to 100% ceramic. A much finer step division can also be selected.

In the represented embodiments, the steps can be made of layers of prefabricated films. In an embodiment variant that is not represented, the electrode 11 can also change the mixing ratio with a continuous gradient.

The production of the flat electrodes illustrated in the figures occurs as follows:

In the embodiment of FIG. 1, a green film made based on a metal and a green film based on a ceramic are produced. The production of these films occurs by stirring a feedstock made of ceramic powder, or metal powder, with a binder, for example, a suitable polymer. A slurry-like feedstock forms which can be spread, for example, by means of a doctor blade at appropriate temperature on a smooth work plate to form a film. After cooling, the material is still flexible but can be handled without carrier and pulled off the work plate. A film with metal material and a film with ceramic material are arranged one on top of the other. From the double layer made of the two layers, the contour of the flat electrode 1 is then cut or punched out. In the process, the hole for the connection wire 4 can also be punched. Subsequently, the flat electrode 1 can be brought to the curved shape represented in FIG. 1, for example, in a compression mold.

The finished green body is then sintered, after the binder has been removed beforehand, for example, thermally, from the feedstock. Subsequently, the connection wire 4 is then mounted and welded, for example, soldered. Lastly, the insulation hose 5 is mounted.

In the case of the bipolar scissors 6 represented in FIG. 2, cutting blades 7 with a layered structure as represented in FIG. 3 are produced. For this purpose, again, green films for the layers 12 and 13 are produced, applied one on top of the other and trimmed. After the sintering, the cutting blades 7 are fastened to a prefabricated commercial scissor handle as represented in FIG. 2 as an example.

In a manner that is not represented, the different flat electrodes are to be connected to a high-frequency voltage source. For this purpose, the connection wire 4 of FIG. 1 or the metal layers 12 of the cutting blades 7 should be brought in contact with corresponding connection lines that either are firmly connected to the electrodes, or, for example, can be plugged in with couplings.

The layered structure shown in FIG. 4 can be produced in principle in a similar way to the one shown in FIG. 3.

In the case of electrode 11 of FIG. 5, the production is similar when the material mixture is to have the step gradients represented. Then, green films that have been stacked one on top of the other in a step structure can be used.

However, if the mixing ratio is to change continuously with the layer height, that is to say without steps, then it is possible to use, for example, scattering techniques in which, at the time of the construction of a feedstock layer, metal and ceramic powder are scattered with gradually changing mixing ratio into the growing feedstock layer.

### LIST OF REFERENCE NUMERALS

1 Electrode head
2 Metal layer
3 Ceramic layer
4 Connection wire
5 Insulation sleeve
6 Scissors
7 Cutting blade
7′ Scissor blade
8 Cutting surface
9 Cutting edge
11 Electrode
12 Metal layer
13 Ceramic layer
14 Insulation layer
15 Metal layer
16 Metal layer

1. Method for producing a flat electrode of a high-frequency surgical instrument, which consists in at least one layer height of metal and in at least one other layer height of ceramic, wherein the flat electrode is produced in the form of a green body and then sintered.

2. Method according to claim 1, wherein the green body is produced from several films which are applied one on top of the other in the form of green films.

3. Method according to claim 2, wherein films made of different materials that alternate one on top of the other are applied.

4. Method according to claim 1, wherein the green body is formed from a mixture of metal and ceramic in which the mixing ratio changes continuously or stepwise with the layer height.

5. Method according to claim 1, wherein the green body is brought to the desired shape before the sintering.

6. Method according to claim 2, wherein the green body is brought to the desired shape before the sintering.

7. Method according to claim 3, wherein the green body is brought to the desired shape before the sintering.

8. Method according to claim 4, wherein the green body is brought to the desired shape before the sintering.
